(19) United States
(12) Patent Application Publication
(10) Pub. No.: US 2014/0037496 A1
(43) Pub. Date: Feb. 6, 2014

(54) CONTROL OF BIO-DECONTAMINATION CYCLES

(71) Applicants: Neil Richard Pomeroy, Shawford (GB); Guy Matthew Turner, Winchester (GB)

(72) Inventors: Neil Richard Pomeroy, Shawford (GB); Guy Matthew Turner, Winchester (GB)

(73) Assignee: BIOQUELL UK LIMITED, Andover, Hampshire (GB)

(21) Appl. No.: 13/980,269

(22) PCT Filed: Jan. 3, 2012
(86) PCT No.: PCT/GB12/50003

§ 371 Date: Aug. 9, 2013

(30) Foreign Application Priority Data
Jan. 18, 2011 (GB) 1100852.1

(51) Int. Cl. A61L 2/24

(52) U.S. Cl. CPC A61L 2/24
USPC 422/3; 422/111

(57) ABSTRACT

This invention relates to improvements in the method of controlling bio-decontamination cycles used for the bio-decontamination of enclosed spaces, such as pharmaceutical clean rooms, isolators and hospital wards. The bio-decontamination cycle comprises a number of phases including at least one gassing phase, during which sterilant vapour is generated and circulated within the enclosed space. The method is characterised by the steps of continuously measuring the modified relative humidity of the air in the enclosed space, the modified relative humidity being the ratio of water and sterilant vapour: capacity of water and sterilant vapour in the air, and using the measured modified relative humidity to control the steps of the process.

### CROSS-REFERENCE TO RELATED APPLICATIONS

This patent application claims priority to

International Application PCT/GB2012/050003 filed Jan. 3, 2012 published as WO 2012/098368, which claims priority to GB 1100852.1 filed Jan. 18, 2011, which applications are incorporated herein by specific reference in their entirety.

### FIELD OF INVENTION

This invention relates to improvements in the method of controlling bio-decontamination cycles used for the bio-decontamination of enclosed spaces, such as pharmaceutical clean rooms, isolators and hospital wards.

### BACKGROUND

Vapour phase bio-decontamination is generally a four phase process. During the first “conditioning” phase the equipment is brought up to working temperature, and in the case of small enclosures the relative humidity inside the enclosed space can be brought to a pre-set value. This is followed by the “gassing” phase during which the active vapour concentration inside the enclosed space is raised. In the “dwell” phase the vapour is distributed inside the enclosed space for a sufficient period of time to ensure that bio-decontamination is achieved. The fourth and final phase is the “aeration” phase in which the active vapour is removed from the enclosed space generally by dilution with clean air.

The most commonly used vapour for bio-decontamination is hydrogen peroxide which is generated by evaporating an aqueous solution of about 30 to 35% w/w. The usual technique for producing a “flash” evaporated vapour is to drop the aqueous solution onto a heated plate held at a temperature above the boiling point of the liquid thus generating a vapour with the same weight ratio as the source liquid. There are two theories as to the action of the hydrogen peroxide; the earlier thinking was that the vapour should be maintained at a concentration below the dew point thus avoiding condensation, the other theory suggests that condensation is necessary to give a rapid bio-decontamination.

There are numerous patents covering the use of gaseous and vapour phase bio-decontamination of enclosed spaces, the most important of which are U.S. Pat. No. 5,173,258, U.S. Pat. No. 7,014,813 and U.S. Pat. No. 7,790,104.

U.S. Pat. No. 5,173,258 describes a single loop closed system in which the carrier gas is circulated from the vapour generator to the chamber to be bio-decontaminated and then back to the vapour generator. On returning to the vapour generator the carrier gas and vapours pass through a device to remove the active vapour and the water vapour thus allowing more hydrogen peroxide to be evaporated into the circulating carrier gas.

U.S. Pat. No. 7,014,813 describes a similar process but has a by-pass loop inside the vapour generator. Thus the vapours are not removed from the circulating carrier gas on returning to the vapour generator during the second and third phases of the cycle. This allows a more rapid build up of vapour concentrations and is normally used in cycles when condensation is required.

In both types of bio-decontamination cycles (in which condensation is to be avoided or encouraged respectively) it is essential that the active vapours are distributed evenly throughout the chamber. In some systems the vapours are delivered from rotating nozzles at high velocities and in others external fans are used to move the vapour mixture around the chamber.

Short cycle time is a key commercial driver for hydrogen peroxide vapour generators. The target assets for bio-decontamination within a hospital are often extremely expensive and hence there is a substantial opportunity cost of closing a facility. Figures that have been produced for the USA suggest that revenues of $5 k per day per bed are not atypical. Consequently, the time saving needs to be maximised while still guaranteeing the efficacy of bio-decontamination.

In the prior art processes, the control of the cycles has been based on monitoring the concentration of the bio-decontaminant to determine when saturation conditions have been reached. However this can lead to misleading results, for example if the space undergoing bio-decontamination contains highly absorbent surfaces, or the space is not properly sealed and fresh air is able to enter.

### DESCRIPTION OF FIGURES

FIG. 1 which shows the concentration of sterilant, preferably hydrogen peroxide (H₂O₂), in the enclosed space (as parts per million (ppm)) against cycle time in minutes.

### DESCRIPTION

In many states of the USA the relative humidity (RH) drops during the winter months to around 5%; the low starting RH means that the time to reach dew point is extended and can lead to unacceptably long cycles. Conversely, many Asian countries experience extremely high relative humidity conditions, with 95% not unheard of. These extremely difficult conditions, due to the rapid onset of condensation, cause control methods based entirely on RH measurements to under-dose and jeopardise efficacy.

It is therefore an object of the present invention to provide a method of control of bio-decontamination cycles which reduces user input and enables the cycle time to be minimised, whilst maintaining the efficacy by bio-decontamination.

The present invention therefore provides a method of controlling a bio-decontamination cycle to decontaminate an enclosed space, said bio-decontamination cycle comprising a number of phases including at least one gassing phase, during which sterilant vapour is generated and circulated within the enclosed space;

characterised by the steps of continuously measuring the modified relative humidity of the air in the enclosed space, the modified relative humidity being the ratio of water and sterilant vapour: capacity of water and sterilant vapour in the air, and using the measured modified relative humidity to control the steps of the process to ensure that condensation occurs.

The basic decontamination process which preferably uses hydrogen peroxide as the sterilant is described in WO-A-2008145990, and is summarised as follows. During the first “conditioning” phase of the decontamination cycle evaporator and nozzle fans of the decontamination apparatus are switched on together with an evaporator heater. This allows the gas generator and the space to be decontaminated to come to a stable temperature. Once thermal stability has been achieved the gas generator moves to a second phase of the decontamination cycle, the “gassing phase”, during which a hydrogen peroxide liquid pump is switched on and hydrogen peroxide solution is “flash” evaporated and mixed with the air leaving the decontamination apparatus.

Once the space has been decontaminated the generator moves to the third “aeration phase” of the cycle. In the aeration phase the hydrogen peroxide liquid pump is switched off, as is the evaporator heater. The evaporator fan is also switched off but an aeration fan is started. The operation of the aeration fan opens flap valves in the apparatus casing and draws in large quantities of air through filters, which decompose the hydrogen peroxide to water and, oxygen and at the same time, absorb the water vapour. The aeration fan is left running to ensure good distribution of the air during aeration. The high air flow generated by the aeration fan reduces the time taken for aeration of the space. Once the hydrogen peroxide vapour concentration within the space to be decontaminated has reached a safe level the generator is switched off.

During further development of this process it has been found, surprisingly, that using “modified relative humidity” (MRH), i.e. the ratio of [water and H₂O₂ vapour] to [capacity for water and H₂O₂ vapour in the air], as the main control parameter is more accurate than using the parameters of the prior art methods. Thus 100% MRH indicates that the air is maximinally saturated with mixed water and H₂O₂ vapours when dew point is reached (whereas 100% RH refers to water vapour only). The method of control of the present invention has been shown to provide 6-log kill of Biological Indicators (“BIs”) using G. stearothermophilus at starting relative humidity between 5 and 95%, i.e. thus compensating for extremes and preventing overgassing which can damage materials and undergassing which leads to ineffective decontamination. Significantly the algorithm used by the method is also capable of adapting to different hydrogen peroxide injection rates resulting from varying power supplies globally.

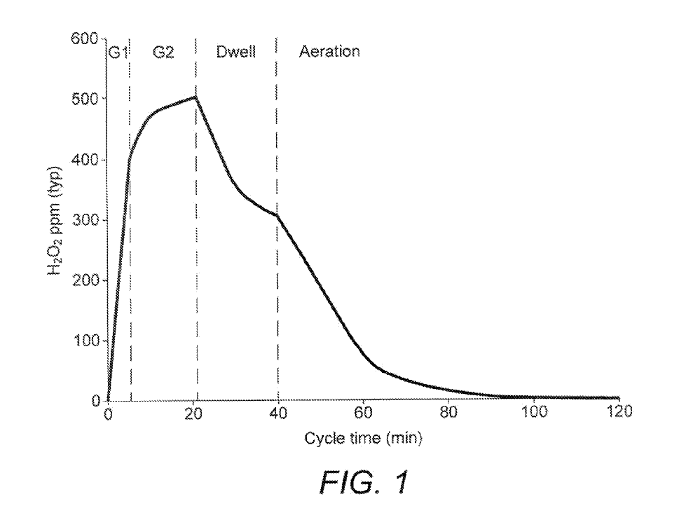

The method of control of the present invention therefore utilizes an algorithm which divides the bio-decontamination process into five distinct phases. This is illustrated in FIG. 1 which shows the concentration of sterilant, preferably hydrogen peroxide (H₂O₂), in the enclosed space (as parts per million (ppm)) against cycle time in minutes.

As described above the first phase is still the “conditioning” phase, during which the vaporiser heats up, and the H₂O₂, relative humidity (RH) and temperature sensors are allowed to stabilise. However, the previously described “gassing” phase is divided into two distinct phases, “G1” and “G2”, which become the second and third phases of the cycle respectively. The gassing commences at the start of G1, during which an H₂O₂ solution is vaporised up to a point where the conditions immediately surrounding the generator are considered to be suitable for bio-decontamination. G2 involves continued gassing such that the entire enclosed space, be it room, chamber or enclosure, is considered to be at a condition suitable for bio-decontamination. The next phase is the “dwell” phase, which optionally involves the cessation of H₂O₂ vaporisation and a fixed time period in which the contaminant may take up the H₂O₂ present and be deactivated. The fifth and final phase is the same “aeration” phase as is described above which involves the catalysis of the H₂O₂ vapour present such that the enclosed space is returned to a condition safe for re-occupation/use.

In order to control the G1 phase a relative humidity sensor capable of measuring both water and H₂O₂ vapour is used, i.e. an atmosphere water content sensor. The measurement of the modified relative humidity (MRH) allows the identification of the point in time where the onset of condensation (dew point) occurs.

The “target MRH” is set at the value to be reached to ensure that condensation occurs and therefore accelerated kill conditions are ensured. The “threshold RH” is set to be the value at which the algorithm changes its approach, i.e. given the high start MRH conditions, it needs to gas longer than it otherwise would in order to compensate for the reduced H₂O₂ concentration in any condensate formed.

Target MRH has been found experimentally to be optimally set at between 70 and 80%.

Threshold RH has been found experimentally to be optimally set at between 80 and 90% of Target MRH, i.e. between 56 and 72% RH.

The reason “target” is in terms of MRH and “threshold” is in terms of RH is that the threshold is only used at the start of the cycle therefore there is no H₂O₂ vapour present, i.e. the two units will be the same.

The end of G1 is defined by reaching the target MRH.

Thus it is the G1 phase that is adapted to compensate for variations in relative humidity and temperature which may occur depending on the location or time of the year etc.

The present method also requires certain other parameters to be pre-set by the user. These are:

1. the volume of the space to be decontaminated (room_volume);

2. whether or not the space is “loaded” or “normal” (cycle type), i.e. an empty room would be normal and a room containing any equipment and/or mattresses or the like providing extra surfaces to be decontaminated, on anything which affects the circulation and/or distribution of the sterilant vapour would be loaded.

Therefore during the conditioning phase, and before vaporisation of H₂O₂ solution commences in the G1 phase, the actual RH and temperature in the space are measured and the process controller performs the following calculations to define the following limits.

First, the controller calculates the theoretical mass of H₂O₂ solution required to be vaporised to reach the target MRH in the enclosed space, using the actual starting RH and temperature.

Secondly, the calculated mass of H₂O₂ solution is multiplied by the volume of the space and the lower gas limit multiplier to give a Lower Limit. This is used to prevent under-gassing in high starting RH environments.

Thirdly, the same calculated mass of H₂O₂ solution is multiplied by the volume of the space and the upper gas limit multiplier to give an Upper Limit. This is used to prevent over-gassing in low starting RH environments.

In environments with high starting RH conditions, the first bead of condensate will be at a lower peroxide concentration than that formed at a lower starting RH. By looking at how close the start RH is to the target value, the system can decide whether to increase the peroxide dosing. Should the system measure and confirm that the start conditions meet this criterion, it decides upon a higher nominal value for G1, and accordingly calculates a higher minimum gassing limit for G1.

The controller then starts the G1 phase and commences the gassing of the H₂O₂ solution (ideally, although not exclusively, at a constant rate) until the Lower Limit is reached. This ensures that the atmosphere is suitable to effectively decontaminate the space. Should the MRH measured at this point exceed the preset target MRH G1 is terminated and G2 is started. Otherwise the vaporisation continues until either the MRH target is met or the Upper Limit is reached.

In this way the controller advantageously adapts to its environments such that neither ineffective nor overly long cycles are brought about by extreme humidity conditions.

The G2 phase is time-based and is a function of the volume and loading of enclosed space to be bio-decontaminated. G2 is thus controlled to allow the H₂O₂ vapour to disperse, having been experimentally confirmed as sufficient to allow full distribution of vapour in an “unloaded” enclosure and therefore sufficient to allow the entirety of said enclosure to reach deactivation conditions. In effect conditions close to the generator exceed deactivation conditions to ensure complete bio-decontamination of the entire enclosure. As such its duration is proportional to the size of the enclosure, such that each cubic metre of volume requires the addition of a specific mass of H₂O₂ vapour.

Should the enclosed space be considered to be loaded the G2 phase time is extended by multiplication by a parameter (loaded factor) to allow for the reduced vapour mobility and more importantly the increased surface area expected.

As this phase is limited by the volume of the enclosed space undergoing decontamination it requires no limits.

Should it be required, injection of H₂O₂ vapour during the dwell phase can also be specified. Otherwise, vaporisation of H₂O₂ ceases and the phase involves a timed countdown until aeration begins.

Preferably in the method of the present invention two distinct phases are calculated and monitored; the first one is concerned with getting up to the required MRH and the second with laying down the condensate. Whilst these phases are preferably run sequentially, they could be run in parallel as one phase in which the target condensate is achieved.

1. A method of controlling a bio-decontamination cycle to decontaminate an enclosed space, said bio-decontamination cycle comprising a number of phases including at least one gassing phase, during which sterilant vapour is generated and circulated within the enclosed space;
characterised by the steps of continuously measuring the modified relative humidity of the air in the enclosed space, the modified relative humidity being the ratio of [water and sterilant vapour]: [capacity of water and sterilant vapour in the air], and using the measured modified relative humidity to control the steps of the process to ensure that condensation occurs.

2. A method as claimed in claim 1 in which there are two gassing phase parameters, the first of which is controlled to compensate for variations in the relative humidity and temperature and the second of which is to compensate for the volume of the enclosed space, and any adverse distribution effects.

3. A method as claimed in claim 1 in which there are two gassing phases.

4. A method as claimed in claim 1 further comprising the step of calculating in which a mass of sterilant solution, which is the theoretical amount of sterilant solution required to be vaporised to reach a preset target modified relative humidity.

5. A method as claimed in claim 4 further comprising the step of calculating a lower limit to prevent under-gassing in high starting relative humidity conditions, and the step of calculating an upper limit to prevent over-gassing in low starting relative humidity conditions,

6. A method as claimed in claim 5 in which the sterilant vapour is generated during the first gassing phase until the first gassing phase is terminated when either the volume of sterilant vapour generated is greater than the upper limit or if the measured relative humidity exceeds a predetermined modified relative humidity conditional on the lower limit having been exceeded.

7. A method as claimed in claim 2 in which the single or multiple gassing phase parameters are modified to compensate for the loading and volume of the enclosed space, which loading is determined by any content of the enclosed space to be decontaminated which affects the circulation and/or distribution of the vapour.

8. A method as claimed in claim 1 in which a relative humidity sensor is used to measure the modified relative humidity.

9. Bio-decontamination apparatus for performing a decontamination cycle comprising means for generating and circulating sterilant vapour and a control module for controlling the bio-decontamination cycle to decontaminate an enclosed space, means for measuring the modified relative humidity of air in an enclosed space, said control module comprising means for performing calculations based on the relative humidity measurement and means for generating control signals to activate or deactivate a gassing phase during which sterilant vapour is generated and circulated within the enclosed space to ensure that condensation occurs.

10. A control module as claimed in claim 9 comprising means to enable a number of parameters to be preset by an operator.

11. A method as claimed in claim 2 in which there are two gassing phases.
